(19) United States
(12) Patent Application Publication
(10) Pub. No.: US 2012/0231042 A1
(43) Pub. Date: Sep. 13, 2012

(54) Pharmaceutical Formulation For Producing Rapidly Disintegrating Tablets

(75) Inventors: Karl Kolter, Limburgerhof (DE); Silke Gebert, Grunstadt (DE); Sandra Kruse, Ober-Florsheim (DE); Michael Schönherr, Frankenthal (DE)

(73) Assignee: BASF SE, Ludwigshafen (DE)

(21) Appl. No.: 13/416,558

(22) Filed: Mar. 9, 2012

Related U.S. Application Data
(60) Provisional application No. 61/450,636, filed on Mar. 9, 2011.

(51) Int. Cl. A61K 9/00; A61K 47/30

(52) U.S. Cl. 424/400; 514/772.3

(57) ABSTRACT

Provided are pharmaceutical formulations in the form of granules comprising
a) about 60-96% by weight of non-film-forming sugars or sugar alcohols,
b) about 1-10% by weight of film-forming sugars or sugar alcohols,
c) about 3-25% by weight of disintegrants,
d) about 0-10% by weight of water-insoluble, film-forming polymers
e) about 0-15% by weight of further pharmaceutically customary auxiliaries where the sum of components a) to e) is 100% by weight. Also provided are methods of making and using the pharmaceutical formulations described herein.

### CROSS-REFERENCE TO RELATED APPLICATIONS

This application claims priority to U.S. Provisional Application No. 61/450,636, filed Mar. 9, 2011, the contents of which are hereby incorporated by reference in their entirety.

### FIELD

The present invention relates to pharmaceutical formulations in the form of granules for producing rapidly disintegrating tablets.

### BACKGROUND

Tablets which rapidly disintegrate and/or rapidly dissolve in the mouth are achieving ever greater importance for the oral administration of drugs. Such tablets have to disintegrate within a short time, at best within 30 seconds, in the oral cavity, have a pleasant taste and should not leave behind a sandy feel. In addition, they should be easy to manufacture, with direct tableting offering considerable advantages over wet granulation, and have high mechanical strength so that they withstand packaging procedures, transportation and also the squeezing out from packs in an undamaged manner.

The products and methods described hitherto only meet these requirements to an inadequate extent, if at all.

Rapidly disintegrating tablets often consist of sugars and sugar alcohols, effervescent systems, microcrystalline cellulose and other water-insoluble fillers such as calcium hydrogenphosphate, cellulose derivatives, corn starch, or polypeptides. Furthermore, water-soluble polymers, customary disintegrants (crosslinked polyvinylpyrrolidone (“PVP”), Na and Ca salt of crosslinked carboxymethylcellulose, Na salt of carboxymethyl starch, low-substituted hydroxypropylcellulose L-HPC) and essentially inorganic water-insoluble constituents (silicas, silicates, inorganic pigments) are used. Furthermore, the tablets can also comprise surfactants.

WO 2003/051338 describes a directly tablettable and readily compressible auxiliary formulation which comprises mannitol and sorbitol. Firstly, an auxiliary premix is prepared by dissolving mannitol and sorbitol in water and subsequently spray-drying (customary spray-drying and SBD methods). Mannitol can additionally be added to this coprocessed mixture. Tablets which additionally comprise disintegrants, release agents, pigment and an active ingredient reportedly disintegrate in the oral cavity within 60 seconds.

US 2002/0071864 A1 describes a tablet which disintegrates in the oral cavity within 60 seconds and is primarily formulated from a physical mixture of spray-dried mannitol and a coarsely granular crosslinked polyvinylpyrrolidone and also a limited selection of active ingredients. These tablets have a breaking strength of ca. 40 N and produce an unpleasant sandy feel in the mouth.

According to U.S. Pat. No. 6,696,085 B2, a methacrylic acid copolymer type C appears to be used as disintegrant. The methacrylic acid copolymer type C is an enteric polymer which is not soluble in the acidic pH range, but is soluble in the pH range of 7, as is present in the oral cavity. Besides a low breaking strength (<20 N), the tablets have a high friability (>7%) and include a high fraction, in the region of 15% by weight, of a coarsely granular disintegrant. Consequently, they have low mechanical strength and, on account of the high fraction of coarsely granular disintegrant, produce an unpleasant sandy feel in the mouth.

EP 0839526 A2 describes a pharmaceutical administration form consisting of an active ingredient, erythritol, crystalline cellulose and a disintegrant. Furthermore, mannitol is incorporated and the disintegrant used is crosslinked polyvinylpyrrolidone, thus giving a physical mixture. The tablets are said to disintegrate in the oral cavity within 60 seconds.

The application JP 2004-265216 describes a tablet which disintegrates in the mouth within 60 seconds and consists of an active ingredient, a water-soluble polyvinyl alcohol-polyethylene glycol copolymer, sugar/sugar alcohol (mannitol) and disintegrant.

WO 2007071581 describes preparations which consist of a sugar or sugar alcohol and crosslinked polyvinylpyrrolidone which are agglomerated with an aqueous dispersion of a water-insoluble polymer. It is a disadvantage of these preparations that tablets produced therefrom experience considerable extensions in the disintegration time upon storage at elevated temperature and elevated humidity. Moreover, the preparations require relatively large amounts of lubricants in the tableting and have a tendency to stick to the punches.

### SUMMARY

One aspect of the invention relates to a pharmaceutical formulation in the form of granules comprising
a) about 60-96% by weight of one or more non-film-forming sugars or sugar alcohols,
b) about 1-10% by weight of one or more film-forming sugars or sugar alcohols,
c) about 3-25% by weight of one or more disintegrants,
d) about 0-10% by weight of one or more water-insoluble, film-forming polymers
e) about 0-15% by weight of one or more pharmaceutically customary auxiliaries wherein the sum of components a) to e) is 100% by weight.

In a specific embodiment, the average particle size of the granules is about 100 μm to 600 μm.

There are many variants of how the specific components can be chosen. In one embodiment, the non-film-forming sugar alcohols comprise mannitol, erythritol, xylitol or mixtures thereof. In yet other embodiments, the film-forming sugar alcohols comprise sorbitol, lactitol, isomalt, maltitol or mixtures thereof. In certain variants, the disintegrants comprise a crosslinked polyvinylpyrrolidone with an average particle size of less than about 50 μm. In yet other embodiments still, the disintegrants comprise a crosslinked polyvinylpyrrolidone with a hydration capacity of greater than 6.5 g/g as disintegrant. In other variants, the pharmaceutically customary substances are selected from one or more of acidifying agents, sweeteners, aromas, flavor enhancers, dyes, binders, thickeners, surfactants, and finely divided pigments.

Additionally, other ranges of the components may be used. Thus, in one embodiment, the formulation comprises granules of
a) about 70-93% by weight of one or more non-film-forming sugars or sugar alcohols,
b) about 2-8% by weight of one or more film-forming sugars or sugar alcohols,
c) about 4-20% by weight of one or more disintegrants,
d) about 0-6% by weight of one or more water-insoluble, film-forming polymers
e) about 0-15% by weight of one or more further pharmaceutically customary auxiliaries.

In other embodiments, the formulation comprises agglomerates of
a) about 80-90% by weight of one or more non-film-forming sugars or sugar alcohols,
b) about 2-6% by weight of one or more film-forming sugars or sugar alcohols
c) about 5-15% by weight of one or more crosslinked polyvinylpyrrolidone,
d) about 1-5% by weight of one or more water-insoluble, film-forming polymers
e) about 0-15% by weight of one or more further pharmaceutically customary auxiliaries.

One or more of the formulations described herein can be used in a tablet. Accordingly, another aspect of the invention relates to a tablet comprising a pharmaceutical formulation described herein, wherein the tablet has a disintegration time of less than about 30 seconds in aqueous milieu. In one or more embodiments, the tablet has a breaking strength of greater than about 50 N. In other embodiments, the tablet comprising about 20 to 99% by weight, based on the total table weight of the pharmaceutical formulation. In yet other embodiments still, the tablet may further comprise auxiliaries.

A third aspect of the invention relates to a method for producing the pharmaceutical formulation described herein. The method comprises agglomerating one or more non-film-forming sugar or sugar alcohol particles and crosslinked polyvinylpyrrolidone with an aqueous solution of the film-forming sugar or sugar alcohol. In one or more embodiments, the crosslinked polyvinylpyrrolidone is in suspended form. In one or more other embodiments, the method further comprises suspending water-insoluble polymers in the aqueous solution.

There are other embodiments of the method where the process conditions and/or environments are varied. For example, in one embodiment, the agglomeration takes place in a fluidized-bed granulator, mixer, paddle dryer or spray tower. In another embodiment, the agglomeration is carried out at a relative exit air humidity of greater than about 85%. In yet another embodiment, the agglomeration is carried out at a relative exit air humidity of greater than about 90%. In other variants, the agglomeration is carried out at a relative exit air humidity of greater than about 95%.

In another variant of this aspect, the agglomeration is carried out in two stages, and in the first stage water is sprayed onto the powder initial charge as a granulating liquid. In a specific embodiment, the agglomeration is carried out at a spraying rate of from about 5 g to 30 g per min per kg of powder initial charge in combination with an inlet air temperature of from about 30 to 60° C. and an exit air temperature of from about 10 to 40° C. In another embodiment, the agglomeration is carried out at a spraying rate of from about 6 g to about 18 g per min per kg of powder initial charge in combination with an inlet air temperature of from about 35 to about 50° C. and an exit air temperature of from about 15 to about 25° C.

### DETAILED DESCRIPTION

Provided herein are orally rapidly disintegrating tablets which leave behind a pleasant feel in the mouth, are mechanically very stable, do not experience disintegration time extensions upon storage at elevated humidity and temperature, and do not exhibit sticking tendencies during tableting.

Accordingly, one aspect of the invention relates to a pharmaceutical preparation which consists of granules comprising
a) about 60-96% by weight of non-film-forming sugars or sugar alcohols,
b) about 1-10% by weight of film-forming sugars or sugar alcohols,
c) about 3-25% by weight of disintegrants,
d) about 0-10% by weight of water-insoluble, film-forming polymers
e) about 0-15% by weight of further pharmaceutically customary auxiliaries where the sum of components a) to e) is 100% by weight.

Furthermore, a method for producing such preparations in the form of granules has been found.

In one or more embodiments of the invention, the granules are suitable for producing orally rapidly disintegrating tablets.

Furthermore, orally rapidly disintegrating tablets comprising such preparations have been found. In one or more embodiments, the tablets disintegrate in the mouth or in an aqueous milieu within about 40 seconds, specifically within about 30 seconds, and more specifically within about 20 seconds.

As used herein, “film-forming” is used to refer to a sugar or sugar alcohol if an aqueous solution, upon drying at 45° C., produces a film-like, smooth covering. Non-film-forming sugars or sugar alcohols exhibit a powdery or crystalline structure after drying.

In one or more embodiments, the pharmaceutical preparations comprise, as component a) about 60-96% by weight of at least one non-film-forming sugar or sugar alcohol, and in specific embodiments about 80 to 90% by weight of non-film-forming sugars, sugar alcohols or mixtures thereof. Suitable non-film-forming sugars or sugar alcohols include, but are not limited to, mannitol, erythritol or xylitol. In one or more embodiments, the sugar or sugar alcohol components are finely divided, with average particle sizes from about 5 to about 100 μm, and in specific embodiments, about 10 to 80 μm. If desired, the particle sizes can be adjusted by grinding.

In specific embodiments, mannitol, xylitol, erythritol or mixtures thereof are used. In an even more specific embodiment, mannitol is used.

In one or more embodiments, as component b), about 1-10% by weight of film-forming sugars or sugar alcohols, and in specific embodiments, about 2-6% by weight of film-forming sugars or sugar alcohols or mixtures thereof are used. The film-forming sugars or sugar alcohols include, but are not limited to: sucrose, glucose, for example in the form of glucose syrup, fructose, lactose, trehalose, sorbitol, maltitol, lactitol, Palatinit® (isomalt) and glucose syrup.

In various embodiments, to determine the film-forming property, 30.0 g of a 15% strength by weight solution of the sugar or sugar alcohol in water are first prepared at 23° C. This solution is poured out onto glass plates with an indentation (dimension: 20 cm×6.5 cm, indentation 0.3 cm) and dried for 4 hours at 45° C. and then assessed visually. Measurement can take place in ambient air. The ambient air here has a relative humidity of about 40% at 23° C. Drying can take place for example, on the heating plate of a film-drawing instrument. A suitable film-drawing instrument is for example a commercial instrument such as the COATMASTER 509 MC.

Products which are present after the drying operation as a clear film or covering are referred to as “film-forming” If crystals are visible and the mass appears powdery, the substances are classified as “non-film-forming”

Accordingly, the following non-limiting examples form a clear film/covering: sorbitol, lactitol, maltitol, palatinit, lactose, sucrose, trehalose or glucose. Non-limiting examples of crystals or a powdery structure are formed by mannitol, xylitol or erythritol.

As component c), disintegrants can be used in amounts of, in some embodiments, from about 3 to 25% by weight, in more specific embodiments, about 4 to 20% by weight, and in even more specific embodiments, about 5 to 15% by weight. Suitable disintegrants include, but are not limited to, crosslinked polyvinylpyrrolidones, sodium croscarmellose, crosslinked Na carboxymethyl starch or L-hydroxypropylcellulose (L-HPC). L-HPC is a low-substituted hydroxypropylcellulose with a degree of substitution of from 5 to 16% (after drying for 1 h at 105° C.). It is also possible to use mixtures of these disintegrants.

In specific embodiments, the disintegrants comprise crosslinked polyvinylpyrrolidones. Such crosslinked polyvinylpyrrolidones are water-insoluble (less than 1 part in 10 000 parts of solvent), but non-film-forming The crosslinked polyvinylpyrrolidone can have an average particle size (volume-average) of from about 2 to 60 μm, in specific embodiments, 2 up to less than 50 μm, and in even more specific embodiments, about 2 up to less than 30 μm. In one embodiment, crosslinked polyvinylpyrrolidones with a hydration capacity of greater than about 6.5 g/g are utilized. Here, the hydration capacity can be determined in accordance with the following method:

2 g of polymer are weighed into a centrifuge tube and left to swell with 40 ml of water for 15 minutes. The mixture is then centrifuged for 15 minutes at 2000 rpm and the supernatant liquid is poured off as completely as possible. The measurement takes place at 20° C.

$$Hydration capacity = \frac{final weight - tare}{initial weight}$$

In one or more embodiments of the invention, the high hydration capacity of the crosslinked polyvinylpyrrolidone in the formulation leads to a very rapid disintegration and produces a particularly soft feel in the mouth.

As component d), water-insoluble film-forming polymers can optionally be used in amounts of from about 0 to 10% by weight, specifically about 0 to 6% by weight, and very specifically about 1 to 5% by weight. In some embodiments, polymers which are insoluble in the pH range from 1 to 14 (i.e. have a pH-independent insolubility in water at every pH) are utilized. In other embodiments, polymers which are insoluble in water at every pH in the pH range from 6 to 14 can also be used.

In one or more embodiments of the invention, the polymers are film-forming polymers. In this context, “film-forming” means that the polymers in aqueous dispersion have a minimum film-forming temperature of from about -20 to +150° C., and in more specific embodiment, about 0 to 100° C.

Suitable polymers include, but are not limited to, polyvinyl acetate, ethylcellulose, methyl methacrylate-ethyl acrylate copolymers, ethyl acrylate-methyl methacrylate-trimethylammonium methyl methacrylate terpolymers or butyl methacrylate-methyl methacrylate-dimethylaminoethyl methacrylate terpolymers.

The acrylate-methacrylate copolymers are described in more detail in the European Pharmacopoeia as Polyacrylate Dispersion 30%, in the USP as Ammonio Methacrylate Copolymer and in JPE as Aminoalkyl-Methacrylate Copolymer E.

In some embodiments, ethylcellulose can be used as component d). in specific embodiments, suitable ethylcelluloses usually have an ethoxyl content of from about 45 to 50%. Such ethylcelluloses, as well as the determination of the ethoxyl content, are described in various pharmacopeias such as e.g. USP 34 (2011). The viscosity of the ethylcellulose measured as 5% strength by weight solution in toluene/ethanol 8:2 is about 3 to 400 mPas, in specific embodiments, about 4 to 200, and in even more specific embodiments, about 5 to 100 mPas. The measurement is carried out here at 25° C. in an Ubbelohde viscometer.

If some variants, by adding pharmaceutically customary auxiliaries (component e) in amounts of from 0 to 15% by weight (e.g., acidifying agents, buffer substances, sweeteners, aromas, flavor enhancers, dyes, etc.), it is possible to further improve the flavor and appearance of the tablets obtained from the formulations. In specific embodiments, one or more of the following substances are used: citric acid, tartaric acid, ascorbic acid, sodium dihydrogenphosphate, cyclamate, saccharin sodium, aspartame, menthol, peppermint aroma, fruit aromas, vanilla aroma, glutamate, riboflavin, betacarotene, water-soluble dyes, and finely divided colored lakes.

By adding water-soluble polymers, in other embodiments, it is possible to further improve the feeling in the mouth by increasing the softness and the feeling of volume. Suitable substances include, for example, polyvinylpyrrolidones, vinylpyrrolidone-vinyl acetate copolymers, polyvinyl alcohols, polyvinyl alcohol-polyethylene glycol graft copolymers, polyethylene glycols, ethylene glycol-propylene glycol block copolymers, hydroxypropylmethylcellulose, hydroxypropylcellulose, hydroxyethylcellulose, carrageenans, pectins, xanthans, and alginates.

Furthermore, surfactants can also be added as components e). Suitable surfactants are, for example, sodium lauryl sulfate, dioctyl sulfosuccinate, alkoxylated sorbitan esters such as polysorbate 80, polyalkoxylated derivatives of castor oil or hydrogenated castor oil, for example Cremophor® RH 40, alkoxylated fatty acids, alkoxylated hydroxy fatty acids, alkoxylated fatty alcohols, alkali metal salts of fatty acids and lecithins.

Furthermore, to further improve the disintegration, finely divided pigments can also be added in one or more embodiments, because they increase the internal interfaces, and water can therefore penetrate more rapidly into the tablet. These pigments, such as iron oxides, titanium dioxide, colloidal or precipitated silica, calcium carbonates, calcium phosphates, naturally have to be very finely divided, otherwise a grainy taste will again arise.

Another aspect of the invention relates to the production of the formulations described herein. Producing the formulations according to one or more embodiments of the invention in the form of granules can take place by build-up agglomeration in mixers, fluidized-bed apparatuses, paddle dryers or spray towers. For this, solid starting materials and granulating liquid can be first brought into contact with one another and the moist material dried simultaneously or subsequently. According to one or more embodiments of the present invention, the granulating liquid used is an aqueous solution of component b), the film-forming sugar or sugar alcohol.

In the case of agglomeration to give granules in the fluidized bed, an aqueous solution of the film-forming sugar or sugar alcohols may be sprayed onto powder initial charge, which is a fluidizing mixture of non-film-forming sugar or sugar alcohol and crosslinked PVP and also optionally further polymers or auxiliaries, as a result of which the fine particles agglomerate.

According to a further embodiment of the invention, a disintegrant, and in specific embodiments, crosslinked PVP is suspended in the aqueous granulating liquid, comprising the film-forming sugars or sugar alcohols.

According to a further embodiment of the invention, insoluble film-forming polymer is suspended in the aqueous granulating liquid comprising the film-forming sugars or sugar alcohols.

According to another embodiment of the invention, a disintegrant, and in specific embodiment crosslinked PVP, and insoluble film-forming polymer are suspended in the aqueous granulating liquid comprising the film-forming sugars or sugar alcohols.

In the case of production in spray towers, specifically so-called FSD or SBD technology (FSD: fluidized spray drying; SBD: spray bed drying) is used. Here, a solution of the non-film-forming sugar or sugar alcohol in water can be first spray-dried in the lower section of the spray dryer or in an attached fluidized bed the addition of crosslinked PVP, and the spraying in of an aqueous solution of the film-forming sugar or sugar alcohol as spray solution takes place, as a result of which the particles agglomerate. Fine particles can also be blown once again in front of the spray nozzle of the sugar or sugar alcohol solution and be additionally agglomerated. In the spray tower, FSD or SBD, a process procedure starting from the crystalline form of the non-film-forming sugar or non-film-forming sugar alcohol is also possible. Here, the crystalline sugar or sugar alcohol is added at the top of the spray tower or in the fines material recycling stream. By spraying an aqueous solution of the film-forming sugar or sugar alcohol, the crystalline solid of the non-film-forming sugar (alcohol) is agglomerated to granules in the tower.

In one or more embodiments, the spray drying of the non-film-forming sugar (alcohol) usually takes place from aqueous solutions having a concentration of from about 10 to 50% by weight.

In other embodiments, the film-forming sugar alcohol is usually used in the form of aqueous solutions having a concentration of from about 1 to 40% by weight. The concentration of the film-forming sugar alcohol in the aqueous spray solution can also be varied in two or more process steps.

For embodiments of the granulation process, the process conditions are chosen such that a relative exit air humidity greater than about 85%, and in specific embodiments, greater than about 90%, and in very specific embodiments, greater than about 95%, with an upper limit of about 99.5% results. A relative exit air humidity of more than about 85% can established by adjusting the spraying rate, the inlet air temperature, the amount of inlet air and the humidity of the inlet air. The two main adjustment parameters are the spraying rate and the inlet air temperature. An increase in the spraying rate, and also a lowering of the inlet air temperature, can lead to an increase in the exit air humidity.

In one or more embodiments of this aspect, the spraying rate can be selected in the range from about 3 g to 35 g per min per kg of powder initial charge, and in specific embodiments, about 5 g to 30 g per min per kg of powder initial charge.

In some embodiments, the inlet air temperatures can be about 20 to 80° C., specifically about 30 to 60° C.

In another embodiment, the exit air temperatures are about 10 to 60° C., specifically about 10 to 40° C. and more specifically about 15 to 30° C.

In some embodiments, the granulation can take place at a spraying rate of from about 5 g to 30 g per min per kg of powder initial charge in combination with an inlet air temperature of from about 30 to 60° C. and an exit air temperature of from about 10 to 40° C. In further embodiments, the granulation takes place at a spraying rate of from about 6 g to 18 g per min per kg of powder initial charge in combination with an inlet air temperature of from about 35 to 50° C. and an exit air temperature of from about 15 to 25° C.

In one or more embodiments described herein, the inlet air humidity can be up to about 99.5% relative humidity.

For the granulation process, it may prove favorable to run a multistage spraying process. Here, the concentration of the aqueous spraying solution of the film-forming sugar or sugar alcohol is varied in two or more steps. At the start, the concentration is kept very low—it can even be about 0% by weight (spraying in of water)—in order to wet the initial charge of materials and to establish a high exit air humidity. During this, no agglomeration to granules starts. Then, the concentration of the aqueous spray solution of the film-forming sugar or sugar alcohol can be increased, as a result of which the initial charge of materials agglomerates to granules. In the individual stages, the process conditions can be varied within the above limits.

Thus, one embodiment relates to a two-stage process in which the spraying rate in the first stage is about 1.5- to 3-times the spraying rate in the second stage. In a further embodiment, a 2-stage process as described above in which, in the first stage, pure water is used as spraying solution. The mass ratio of the spraying solutions of the first and the second stage can be varied from about 1:5 to 5:1, and in specific embodiments, about 1:2 to 2:1.

The granule size can be controlled via the amount of film-forming sugar or sugar alcohol in the product. The more of this substance which is introduced, generally, the larger the granules.

Further adjustment parameters for the granule size that can be considered include the fineness of the spray droplet of the binder solution (adjustable via the pressure of the atomizing gas), the nozzle geometry and the distance between the nozzle and the product bed. The finer and more uniform the spraying is carried out, the finer and more uniform are the resulting granules. The further the nozzle from the product bed, generally, the worse the agglomeration behavior. Usually, the pressure of the atomizing gas is in the range from about 0.5 to 5 bar, and specifically about 1.0 to 3.0 bar. The nozzles used are generally two-substance nozzles. The nozzle diameters are usually in the range from about 0.5 to 4.0 mm, specifically about 0.8 to 3.0 mm The distance between the nozzles and the product bed is governed by the size of the atomization apparatus. What distances are suitable in an individual case can be ascertained by the person skilled in the art by a few customary experiments.

When the spraying process is complete, the moist material is dried. For this, the inlet air temperature can increased in order to speed up the drying process. Customary temperatures here are about 30-100° C. During the drying phase, it may be advantageous to reduce the amount of inlet air and to thereby prevent abrasion of the granules by a high mechanical stress.

In the case of production in a paddle dryer, the initial charge of materials can be sprayed with a solution of the film-forming sugar or sugar alcohol in water and dried simultaneously or subsequently.

Furthermore, the granules can also be produced in a mixer by means of a continuously operated mixing aggregation. One example of such a continuously operated form of mixing aggregation is so-called “Schugi granulation”. In this process, in a continuously operating vertically arranged high-speed mixer, solid starting materials and the granulating liquid comprising the film-forming sugar or sugar alcohol are intensively mixed together (see also M. Bohnet, “Mechanische Verfahrenstechnik” [Mechanical process engineering], Wiley VCH Verlag, Weinheim 2004, p. 198 ff.).

The granules produced in this way have an average particle size (volume-average) of about 100-600 μm, in specific embodiments about 120-500 μm and in more specific embodiments of about 140-400 μm.

The film-forming sugar or sugar alcohol serves here as agglomerating agent in order to agglomerate the fine non-film-forming sugar or sugar alcohol crystals and the particles of crosslinked PVP.

The granules according to one or more embodiments of the invention can be tabletted with small amounts of lubricants; this does not result in any significant influence on the disintegration time. In addition, the granules according to one or more embodiments of the invention do not stick to the punches, as a result of which higher tableting speeds and shorter tableting processes are achieved.

The formulations according to one or more embodiments the invention are suitable for producing orally rapidly disintegrating tablets.

Thus, another aspect of the invention relates to tablets comprising one or more formulations described herein. The formulations according to one or more embodiments of the invention can advantageously also be used for producing tablets which are left to disintegrate in a glass of water prior to use. The production of tablets which are swallowed intact is also of course possible.

For producing tablets, the customary methods can be used, with direct tableting and roll compaction offering particular advantages. On account of the special properties of the formulations according to one or more embodiments of the invention, in some embodiments, all that is required are an active ingredient, a formulation according to one or more embodiments of the invention and a lubricant. The tablet formulation according to this aspect is thus very simple, very reproducible and the method can be easily validated.

Surprisingly, it has been found that a film-forming sugar or sugar alcohol can act as a very effective binder in the event of appropriate process control; this moreover considerably increases the rate of decomposition of tablets. In this way, it is possible to produce granules which comprise neither a water-soluble polymer nor a water-insoluble polymer as binder. The use of water-soluble polymers leads to significantly longer disintegration times of tablets and the use of water-insoluble polymers. Although it does produce tablets which are rapidly disintegrating, they have considerable disintegration time extensions upon storage at elevated temperature and/or increased humidity.

The disintegration time of the preparations according to one or more embodiments of the invention is generally not influenced by increased temperature and humidity.

In addition, the preparations according to one or more embodiments of the invention have extraordinarily good flowabilities and compressibilities which lead to mechanically very stable tablets. The breaking strength of the tablets produced with the help of the pharmaceutical formulations according to one or more embodiments of the invention is greater than about 50 N. In other embodiments, the breaking strengths are often above about 80 N, even using active ingredients that are difficult to compress. The friabilities are less than about 0.2% in one or more embodiments. Consequently, damage during customary tablet handling generally does not arise.

On account of the fine crosslinked polyvinylpyrrolidone, the tablets according to one or more embodiments of the invention exhibit virtually no changes in the tablet surface upon storage in damp conditions. In contrast to coarse crosslinked polyvinylpyrrolidone, there is generally no pimpling due to severely swollen particles. The formulations according to one or more embodiments of the invention are therefore very stable upon storage and retain their attractive appearance.

As mentioned, the granules are produced in one or more embodiments at exit air humidities of at least about 85% relative humidity. Such exit air humidities are entirely non-customary since conventional formulations completely clump together at such exit air humidities and can no longer be fluidized and processed. Surprisingly, it has been found that below about 85% relative humidity, no satisfactory granulation takes place, but above about 85% very uniform granules are formed and the process can be run without problems.

### EXAMPLES

Film Formation Test:

To determine the film-forming property, firstly, at 23° C., 30.0 g of a 15% strength by weight solution of the sugar or sugar alcohol in water were prepared. This solution was poured onto glass plates with an indentation (dimension: 20 cm×6.5 cm, indentation 0.3 cm) and dried at 45° C. for 4 hours and then assessed visually. The ambient air here had a relative humidity of 40% at 23° C. Drying was carried out on a heating plate of the film-drawing instrument COATMASTER 509 MC (Erichsen).

| Sugar (alcohol) | Clear film | Notes |
|---|---|---|
| Maltitol | Yes | Film-forming |
| Lactitol | Yes | Film-forming |
| Sorbitol | Yes | Film-forming |
| Palatinit | Yes | Film-forming |
| Glucose syrup DE 61 | Yes | Film-forming |
| Xylitol | No | Crystals; non-film-forming |
| Mannitol | No | Crystals; non-film-forming |
| Erythritol | No | Crystals; non-film-forming |

The particle size determinations listed below were carried out using a Malvern Mastersizer.

Examples A-H show the agglomerating effect of film-forming sugars or sugar alcohols.

Non-film-forming sugar/sugar alcohol and crosslinked PVP and also further auxiliaries were introduced as initial charge in the fluidized bed and agglomerated with a 3% strength by weight aqueous solution of the film-forming sugar or sugar alcohol, which were sprayed into the fluidized-bed granulator (Glatt, GPCG 3.1) by means of topspray methods.

The following production conditions were used:

Batch size: 2.5 kg

Concentration of the binder solution/dispersion: 3% by weight

Inlet air temperature: 60 ° C.

Exit air temperature: 18-25° C.

Spraying rate 70 g/min

In all cases, a relative humidity of the exit air of greater than 90% was achieved.

TABLE 1
Formulation composition of granules A to H in % by weight

| | A | B | C | D | E | F | G | H | I |
|---|---|---|---|---|---|---|---|---|---|
| Xylitol | 85 | — | — | — | — | — | — | — | 82 |
| Mannitol | — | 87 | 87 | 87 | — | 83 | 85 | 85 | — |
| (Pearlitol ® 50 C) | | | | | | | | | |
| average particle size 50 μm | | | | | | | | | |
| Erythritol | — | — | — | — | 84 | — | — | — | — |
| (Eridex ® 16952) ground | | | | | | | | | |
| average particle size 40 μm | | | | | | | | | |
| Crosslinked PVP | 12 | 10 | 10 | 10 | 12 | 12 | 10 | 10 | 10 |
| (Kollidon ® CL-SF) | | | | | | | | | |
| average particle size 17 μm | | | | | | | | | |
| Sorbitol | 3.0 | 3.0 | — | — | — | 5.0 | 3.0 | 3.0 | — |
| Maltitol | — | — | 3.0 | — | 4.0 | — | — | — | 3.0 |
| Lactitol | — | — | — | 3.0 | — | — | — | — | — |
| Polyvinyl alcohol/polyethylene | — | — | — | — | — | — | 2 | — | — |
| glycol block copolymer | | | | | | | | | |
| (Kollicoat ® IR) | | | | | | | | | |
| Methacrylic acid/ethyl acrylate | — | — | — | — | — | — | — | — | 5.0 |
| copolymer | | | | | | | | | |
| (Kollicoat ® MAE 100 P) | | | | | | | | | |
| Ethylcellulose | — | — | — | — | — | — | — | 2.0 | — |

Kollicoat® MAE 100 P: powder, 97% by weight methacrylic acid-ethyl acrylate copolymer (1:1), 2.3% by weight polysorbate 80, 0.7% by weight sodium lauryl sulfate Ethylcellulose: 7 mPas, ethoxyl content 48.0-49.5% Kollicoat® IR: graft copolymer of 25% by weight of PEG6000 and 75% by weight of polyvinyl alcohol side chains, MW 45 000 D

The granules produced had the following properties:

| | Average particle size | |
|---|---|---|
| | [μm] | Angle of repose [°] |
| A | 269 | 31 |
| B | 241 | 31 |
| C | 254 | 31 |
| D | 235 | 31 |
| E | 284 | 30 |
| F | 340 | 29 |
| G | 301 | 30 |
| H | 248 | 31 |
| I | 267 | 31 |

The particle size was determined by means of light scattering and the average particle size (volume-average) used was the D(4,3) value.

The granules produced in this way were mixed with 2.0% by weight of lubricant (sodium stearyl fumarate) in a Turbula mixer for 5 min. These mixtures were then tabletted on a fully instrumented rotary tableting press (Korsch XL 100/8) at a rotational speed of 40 rpm. The rotary tableting press was equipped with 8 punches (10 mm, biplane, faceted). The tablet weight was adjusted to 300 mg. The pressing force was adjusted such that the breaking strength of the tablets was 50 N.

The tablets were analyzed with regard to breaking strength (tablet tester HT-TMB-Cl-12 F, Kraemer), friability (Roche friabilator, Erweka) and disintegration time in phosphate buffer pH 7.2 (disintegration tester ZT 74, Erweka).

TABLE 2
Tablet properties formulations A to I

| | | | | Disintegration | |
|---|---|---|---|---|---|
| | Breaking | Fria- | Disintegration | time [s] | Disintegration |
| | strength | bility | time [s] | 40° C./75% | time [s] |
| | [N] | [%] | initial | r.h. | 60° C. |
| A | 60 | 0.10 | 39 | 40 | 41 |
| B | 60 | 0.10 | 18 | 18 | 19 |
| C | 60 | 0.10 | 17 | 19 | 18 |
| D | 60 | 0.10 | 17 | 16 | 19 |
| E | 60 | 0.10 | 36 | 38 | 39 |
| F | 60 | 0.10 | 28 | 30 | 27 |
| G | 60 | 0.10 | 28 | 29 | 30 |
| H | 60 | 0.10 | 16 | 17 | 18 |
| I | 60 | 0.10 | 16 | 18 | 16 |

### Comparative Examples

### Example J (Comparative Example to B)

Agglomeration of mannitol with a non-film-forming sugar

Production was carried out analogously to Example B, but using a xylitol solution instead of a sorbitol solution.

Virtually no agglomeration took place. The average particle size was 97 μm and the angle of repose 44°. Tableting was not possible due to the poor flowability.

### Example K (Comparative Example to B)

Agglomeration at a relative humidity of 70% (less than 85%)

Production was carried out analogously to Example B, except the spraying rate was adjusted such that a relative exit air humidity of 70% resulted.

Virtually no agglomeration took place. The average particle size was 89 μm and the angle of repose 43° . Tableting was not possible due to poor flowability.

### Example L (Comparative Example to Example H of WO 2007/071581)

Agglomeration of mannitol with a non-water-soluble film-forming polymer

Production was carried out analogously to Example H of the above-cited laid-open specification. The disintegration time of the tablets directly after production was 30 s. The tablets were then stored at 40° C./75% r.h. and at 60° C. for 4 weeks. During this time, the disintegration time increased to 180 s and 204 s, respectively.

### Examples M-P

Examples M to P show the suitability of a rapidly disintegrating auxiliary in an active ingredient formulation.

The rapidly disintegrating auxiliary is produced by agglomeration in the fluidized bed analogously to Example C. The direct tableting composition produced in this way was mixed with active ingredient and 0.5 to 1.0% by weight of lubricant (Mg stearate) and then compressed on a rotary tableting press (Korsch PH 100/6) to give tablets with a breaking strength of 50 N.

TABLE 3
Active ingredient, amount of active ingredient, tablet weight
and diameter of the active ingredient formulations M-P

| | | Amount of active | | |
|---|---|---|---|---|
| | Active ingredient | ingredient | Tablet weight | Diameter |
| M | Famotidine | 20 mg | 250 mg | 8 mm |
| N | Desloratadine | 2 mg | 100 mg | 6 mm |
| O | Sumatriptan | 25 mg | 280 mg | 10 mm |
| P | Ondansetron | 4 mg | 120 mg | 7 mm |

The tablets were investigated as regards breaking strength (table tester HT-TMB-Cl-12 F, Kraemer), friability (Roche friabilator, Erweka) and disintegration time in phosphate buffer pH 7.2 (disintegration tester ZT 74, Erweka).

TABLE 4
Tablet properties formulations J to M

| | Breaking strength | | |
|---|---|---|---|
| | [N] | Friability [%] | Disintegration time [s] |
| M | 51 | <0.20 | 30 |
| N | 50 | <0.20 | 18 |
| O | 52 | <0.20 | 21 |
| P | 50 | <0.20 | 20 |

### Examples Q to S

Non-film-forming sugar/sugar alcohol and crosslinked PVP and also further auxiliaries were introduced as initial charge in the fluidized bed and agglomerated firstly with demineralized water and then with a 6% strength by weight aqueous solution of the film-forming sugar or sugar alcohol, which was sprayed into the fluidized-bed granulator (Glatt, GPCG 3.1) by means of topspray methods.

The following production conditions were used:

Batch size: 2.5 kg

Concentration of the binder solution/dispersion: 6% by weight

Inlet air temperature: 45 ° C.

Exit air temperature: 16-23° C.

Spraying rate of water 150 g/min

Spraying rate of binder solution 60 g/min

Amount of water 1.25 kg

Amount of binder solution 1.25 kg

In all cases, a relative humidity of the exit air of greater than 90% was achieved.

TABLE 1
Formulation composition of granules Q to S in % by weight

| | Q | R | S |
|---|---|---|---|
| Mannitol | 83 | 85.0 | 88.5 |
| (Pearlitol ® 50 C) | | | |
| average particle size 50 μm | | | |
| Crosslinked PVP | 12 | 10 | 8 |
| (Kollidon ® CL-F) | | | |
| average particle size 30 μm | | | |
| Sorbitol | 3.0 | — | — |
| Maltitol | — | 3.5 | — |
| Lactitol | — | — | 2.5 |
| Ethylcellulose, fine powder, | 2.0 | 1.5 | 1.0 |
| 7 mPas, 48% by weight ethoxyl | | | |
| groups | | | |

The granules produced had the following properties:

| | Average particle size | |
|---|---|---|
| | [μm] | Angle of repose [°] |
| Q | 283 | 30 |
| R | 305 | 30 |
| S | 325 | 31 |

The particle size was ascertained by means of light scattering and the average particle size (volume-average) used was the D(4,3) value.

1. A pharmaceutical formulation in the form of granules comprising
a) about 60-96% by weight of one or more non-film-forming sugars or sugar alcohols,
b) about 1-10% by weight of one or more film-forming sugars or sugar alcohols,
c) about 3-25% by weight of one or more disintegrants,
d) about 0-10% by weight of one or more water-insoluble, film-forming polymers
e) about 0-15% by weight of one or more pharmaceutically customary auxiliaries wherein the sum of components a) to e) is 100% by weight.

2. The formulation of claim 1, wherein the average particle size of the granules is about 100 μm to 600 μm.

3. The formulation of claim 1, wherein the non-film-forming sugar alcohols comprise mannitol, erythritol, xylitol or mixtures thereof.

4. The formulation of claim 1, wherein the film-forming sugar alcohols comprise sorbitol, lactitol, isomalt, maltitol or mixtures thereof.

5. The formulation of claim 1, wherein the disintegrants comprise a crosslinked polyvinylpyrrolidone with an average particle size of less than about 50 μm.

6. The formulation of claim 1, wherein the disintegrants comprise a crosslinked polyvinylpyrrolidone with a hydration capacity of greater than 6.5 g/g.

7. The formulation of claim 1, wherein the pharmaceutically customary substances are selected from one or more of acidifying agents, sweeteners, aromas, flavor enhancers, dyes, binders, thickeners, surfactants, and finely divided pigments.

8. The formulation of claim 1, comprising granules of
a) about 70-93% by weight of one or more non-film-forming sugars or sugar alcohols,
b) about 2-8% by weight of one or more film-forming sugars or sugar alcohols,
c) about 4-20% by weight of one or more disintegrants,
d) about 0-6% by weight of one or more water-insoluble, film-forming polymers
e) about 0-15% by weight of one or more further pharmaceutically customary auxiliaries.

9. The formulation of claim 1, comprising agglomerates of
a) about 80-90% by weight of one or more non-film-forming sugars or sugar alcohols,
b) about 2-6% by weight of one or more film-forming sugars or sugar alcohols
c) about 5-15% by weight of one or more crosslinked polyvinylpyrrolidone,
d) about 1-5% by weight of one or more water-insoluble, film-forming polymers
e) about 0-15% by weight of one or more further pharmaceutically customary auxiliaries.

10. A tablet comprising the pharmaceutical formulation of claim 1, wherein the tablet has a disintegration time of less than about 30 seconds in aqueous milieu.

11. The tablet of claim 10, wherein the tablet has a breaking strength of greater than about 50 N.

12. The tablet of claim 10, wherein the tablet comprising about 20 to 99% by weight, based on the total table weight of the pharmaceutical formulation of claim 1.

13. The tablet of claim 10, further comprising auxiliaries.

14. A method for producing the pharmaceutical formulation of claim 1, the method comprising agglomerating one or more non-film-forming sugar or sugar alcohol particles and crosslinked polyvinylpyrrolidone with an aqueous solution of the film-forming sugar or sugar alcohol.

15. The method of claim 14, wherein the crosslinked polyvinylpyrrolidone is in suspended form.

16. The method of claim 15, further comprising suspending water-insoluble polymers in the aqueous solution.

17. The method of claim 14, wherein the agglomeration takes place in a fluidized-bed granulator, mixer, paddle dryer or spray tower.

18. The method of claim 14, wherein the agglomeration is carried out at a relative exit air humidity of greater than about 85%.

19. The method of claim 18, wherein the agglomeration is carried out at a relative exit air humidity of greater than about 90%.

20. The method of claim 19, wherein the agglomeration is carried out at a relative exit air humidity of greater than about 95%.

21. The method of claim 14, wherein the agglomeration is carried out in two stages, and in the first stage, water is sprayed onto the powder initial charge as a granulating liquid.

22. The method of claim 21, wherein the agglomeration is carried out at a spraying rate of from about 5 g to 30 g per min per kg of powder initial charge in combination with an inlet air temperature of from about 30 to 60° C. and an exit air temperature of from about 10 to 40° C.

23. The method of claims 22, wherein the agglomeration is carried out at a spraying rate of from about 6 g to about 18 g per min per kg of powder initial charge in combination with an inlet air temperature of from about 35 to about 50° C. and an exit air temperature of from about 15 to about 25° C.
